(19) United States
(12) Patent Application Publication
(10) Pub. No.: US 2008/0255619 A1
(43) Pub. Date: Oct. 16, 2008

(54) Posterior spinal fixation with colinear facet screw

(76) Inventors: Gary A. Schneiderman, Sacramento, CA (US); Bradley J. Glenn, Oneida, WI (US)

Correspondence Address: BRADLEY P. HEISLER;HEISLER & ASSOCIATES, 3017 DOUGLAS BOULEVARD, SUTIE 300, ROSEVILLE, CA, 95661, US

(21) Appl. No.: 12/082,421

(22) Filed: Apr. 10, 2008

Related U.S. Application Data
(60) Provisional application No. 60/907,575, filed on Apr. 10, 2007.

(51) Int. Cl. A61B 17/58; A61B 17/56

(52) U.S. Cl. 606/276; 606/246; 606/301

(57) ABSTRACT

A three element spinal fixation system for implantation from a posterior lateral incision. The first element in the form of a translaminar cannula is adapted to be implanted along a longitudinal axis of a lamina of a vertebra. A bracket is provided as a second element with a first end removably secured to the translaminar cannula and the second end secured to vertebra inferior to an adjacent vertebra in which the translaminar cannula is implanted. The translaminar cannula has a hollow central bore which receives a third element in the form of a translaminar facet screw. The translaminar facet screw is sufficiently long to be able to pass through the central bore and out of the translaminar cannula, passing through the facet joint to fix the two vertebra at the facet joint. Both contralateral and ipsilateral fixation are thus provided with all three elements implanted from a single ipsilateral incision.

### CROSS-REFERENCE TO RELATED APPLICATIONS

This application claims benefit under Title 35, United States Code §119(e) of U.S. Provisional Application No. 60/907,575 filed on Apr. 10, 2007.

### FIELD OF THE INVENTION

The following invention relates to devices implantable within the body to affix adjacent vertebra together, such as during a spinal fusion procedure or other procedure where fixation of adjacent vertebra is required. More particularly, this invention relates to spinal fixation systems which are implanted from a posterior or posterior lateral incision site and which feature a facet screw for securing adjacent vertebra together.

### BACKGROUND OF THE INVENTION

Surgical treatment of back pain frequently necessitates fixation of the posterior elements of the spine, often in combination with anterior interbody fusion. Most typically, fixation of the posterior elements of the spine occurs through utilization of elongate rods oriented parallel and lateral to the spine and secured to the spine through pedicle screws, and often also including crosslinks between the rods. The anterior interbody fusion typically includes an interbody implant into the disk space and bone graft within this disk space to fuse the adjacent vertebra together. Anterior interbody fusion is increasingly being performed from a posterior-lateral approach. Such a procedure is commonly referred to as transforaminal lumbar interbody fusion or “TILF.”

With known prior art posterior spinal fixation systems, such as rods and pedicle screws, incisions are required to approach both sides of the spine. It is generally beneficial to minimize the number and size of incisions required for a surgical procedure to aide in recovery for the patient and minimize the potential for complications.

One known alternative to use of spinal rods and pedicle screws for posterior fixation is to utilize facet screws which pass from an ipsilateral side of the spinous process through the contralateral lamina along an elongate dimension of the contralateral lamina and then through the contralateral facet joint between the two adjacent vertebra. Such facet screws and their method of utilization to fix adjacent vertebra are described in U.S. Published Patent Application No. 2007/0055236 to Hudgins. Hudgins teaches use of two such facet screws passing in two opposite directions. Thus, two incisions are still involved to provide this spinal fixation.

When a single facet screw is utilized, fixation occurs only on the contralateral side of the spine, leaving the ipsilateral side of the spine less than fully fixed. Other prior art devices for fixation of facets of adjacent vertebra are described in U.S. Pat. Nos. 6,648,893 and 5,527,312. Such devices require multiple incisions and the implantation of devices from both ipsilateral and contralateral sides of the spine to achieve full spinal fixation. Accordingly, a need exists for a spinal fixation system which can fully fix both ipsilateral and contralateral sides of adjacent vertebra together through a single posterior lateral incision.

### SUMMARY OF THE INVENTION

With this invention a fixation system is provided which can affix both ipsilateral and contralateral sides of a superior vertebra to ipsilateral and contralateral sides of an inferior vertebra with the various components of the fixation system each implanted through a single posterior lateral incision. The ipsilateral side is the side of each vertebra closest to the incision and implantation site, with the contralateral side opposite the incision and implantation site. The superior vertebra is the more cephalad vertebra of the two, with the inferior vertebra more caudal. The single posterior lateral incision used for implantation of the elements of the fixation system can also be used for interbody fusion in a disk space between the superior vertebra and the inferior vertebra. The fixation system utilizes three separate elements in the preferred embodiment, with the first element in the form of a translaminar cannula, the second element in the form of a bracket and the third element in the form of a translaminar facet screw.

The translaminar cannula is preferably elongate and threaded on an outer surface and has a hollow bore passing from a proximal end to a distal end. The threaded translaminar cannula is preferably implanted from an ipsilateral side of the spinous process, passing into the vertebra at a portion of the ipsilateral lamina or a portion of the spinous process, and along a line which is colinear with a central portion of a longitudinal axis of the contralateral lamina. The threaded translaminar cannula preferably includes a shoulder which abuts the superior vertebra surface after the translaminar cannula has been fully implanted. Portions of the translaminar cannula also extend proximally from the shoulder.

The bracket is configured to include a first end and a second end with the first end attachable to the translaminar cannula. Most preferably, this attachment is achieved by forming the first end of the bracket to include a pair of arms spaced apart similar to a diameter of portions of the translaminar cannula proximal to the shoulder. These arms are placed adjacent the shoulder and on a proximal side of the shoulder. A nut is then threaded onto the proximal end of the translaminar cannula and tightened down until it traps the arms of the first end of the bracket against the shoulder. A second end of the bracket preferably includes a hook which can wrap around a portion of the inferior vertebra. Most preferably, this portion of the inferior vertebra is on an ipsilateral side of the inferior vertebra for fixation of the ipsilateral side of the inferior vertebra to the ipsilateral side of the superior vertebra. As an alternative to the hook, the second end could include a hole for receiving a laminar screw or can bend to place a hole at the second end of the bracket adjacent the pedicle of the inferior vertebra for fixation to the inferior vertebra with a pedicle screw.

The translaminar facet screw is preferably configured to pass through the bore in the translaminar cannula. The translaminar facet screw is longer than the translaminar cannula and extends from a head at a first end to a tip at a second end. Portions of the translaminar facet screw adjacent the tip are threaded with threads or other engagement means configured to engagement with bone in the spine, and in particular inferior vertebra portions of the contralateral facet joint. This contralateral facet joint is aligned axially with the long axis of the translaminar cannula so that the translaminar facet screw is aligned by the translaminar cannula for passage through the facet joint and fixation of the contralateral facet joint between the inferior vertebra and the superior vertebra. Thus, the translaminar facet screw provides fixation between the superior vertebra and the inferior vertebra on the contralateral side of the spine.

### OBJECTS OF THE INVENTION

Accordingly, a primary object of the present invention is to provide an implantable fixation system for fixing adjacent vertebra together.

Another object of the present invention is to provide a spinal fixation system which fixes adjacent vertebra together with separate components each implantable through a common posterior lateral incision.

Another object of the present invention is to provide a method for fixing a superior vertebra to an inferior vertebra through a single posterior lateral incision.

Another object of the present invention is to provide a spinal fixation system which is implantable in a minimally invasive procedure.

Another object of the present invention is to provide a spinal fixation system which can be implanted in a highly reliable fashion and which exhibits a low instance of failure.

Another object of the present invention is to provide a spinal fixation system which has separate components which can each be implanted through a common incision through which anterior interbody fusion is also accomplished.

Other further objects of the present invention will become apparent from a careful reading of the included drawing figures, the claims and detailed description of the invention.

### BRIEF DESCRIPTION OF THE DRAWINGS

FIG. 1 is a perspective view of a threaded translaminar cannula providing a first element of the fixation system of this invention according to a preferred embodiment.

FIG. 2 is a front elevation view of that which is shown in FIG. 1.

FIG. 3 is a top plan view of that which is shown in FIG. 1.

FIG. 4 is a front elevation full sectional view of that which is shown in FIG. 1.

FIG. 5 is a perspective view of a bracket providing a second element of the fixation system of this invention according to a preferred embodiment.

FIG. 6 is a perspective view of an alternative bracket providing an alternative to the bracket of FIG. 5.

FIG. 7 is a perspective view of a second alternative bracket providing a second alternative to the bracket of FIG. 5.

FIG. 8 is a perspective exploded parts view of the threaded translaminar cannula and bracket, along with a nut and illustrating how these separate components are attachable together according to this invention.

FIG. 9 is a perspective view similar to that which is shown in FIG. 8, but after attachment of the nut and bracket to the threaded translaminar cannula.

FIG. 10 is a perspective view similar to that which is shown in FIG. 9, but with the second alternative bracket of FIG. 7 shown along with a pedicle screw.

FIG. 11 is a perspective view of a translaminar facet screw providing a third element of the fixation system of this invention according to a preferred embodiment.

FIG. 12 is a perspective view of the translaminar facet screw in the process of being inserted into the threaded translaminar cannula according to a method of this invention.

FIG. 13 is a perspective view similar to that which is shown in FIG. 12, but after completion of insertion of the translaminar facet screw into the threaded translaminar cannula.

FIG. 14 is a perspective view of an alternative threaded translaminar cannula and an alternative translaminar facet screw in the process of being inserted into the alternative translaminar cannula.

FIGS. 15-20 are perspective views of a pair of adjacent vertebrae and illustrating sequential steps in the process of implanting the threaded translaminar cannula, the bracket, the alternative bracket and the translaminar facet screw into various different portions of the vertebrae and secured to each other to achieve fixation of the two vertebrae together according to a method of this invention.

### DESCRIPTION OF THE PREFERRED EMBODIMENT

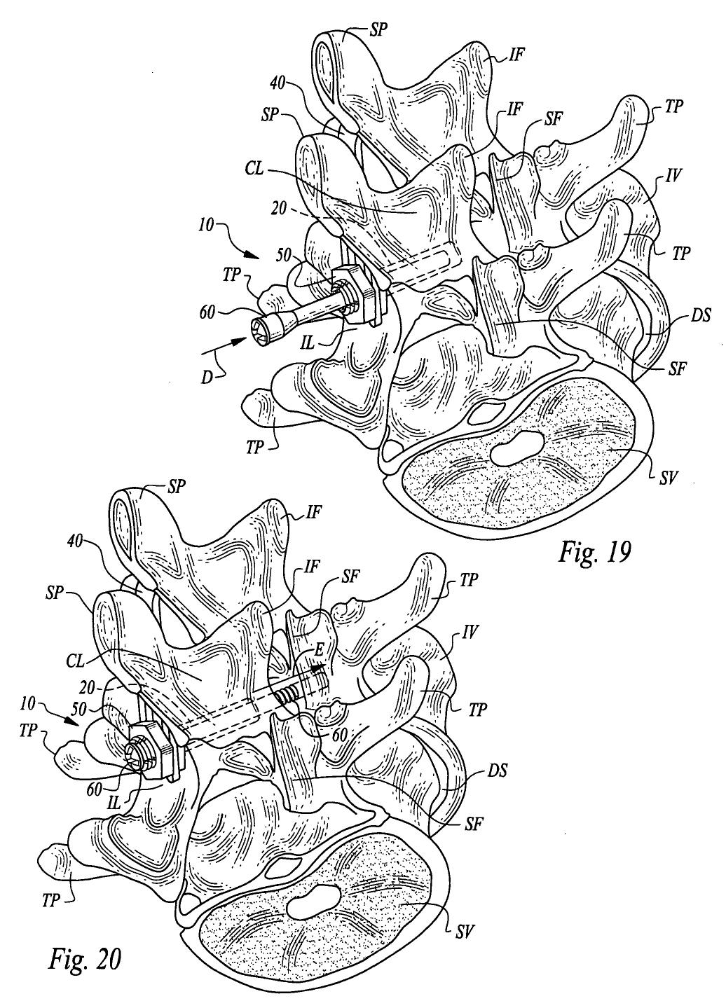

Referring to the drawings, wherein like reference numerals represent like parts throughout the various drawing figures, reference numeral 10 (FIGS. 19 and 20) is directed to a fixation system for fixing a pair of vertebrae, illustrated herein as a superior vertebra SV and an inferior vertebra IV, and with the elements of the fixation system 10 each implantable from a single posterior lateral incision. This same posterior lateral incision can also be used for fusion of the superior vertebra SV and the inferior vertebra IV together, such as by appropriate fusion within the disk space DS on the anterior side of the spine.

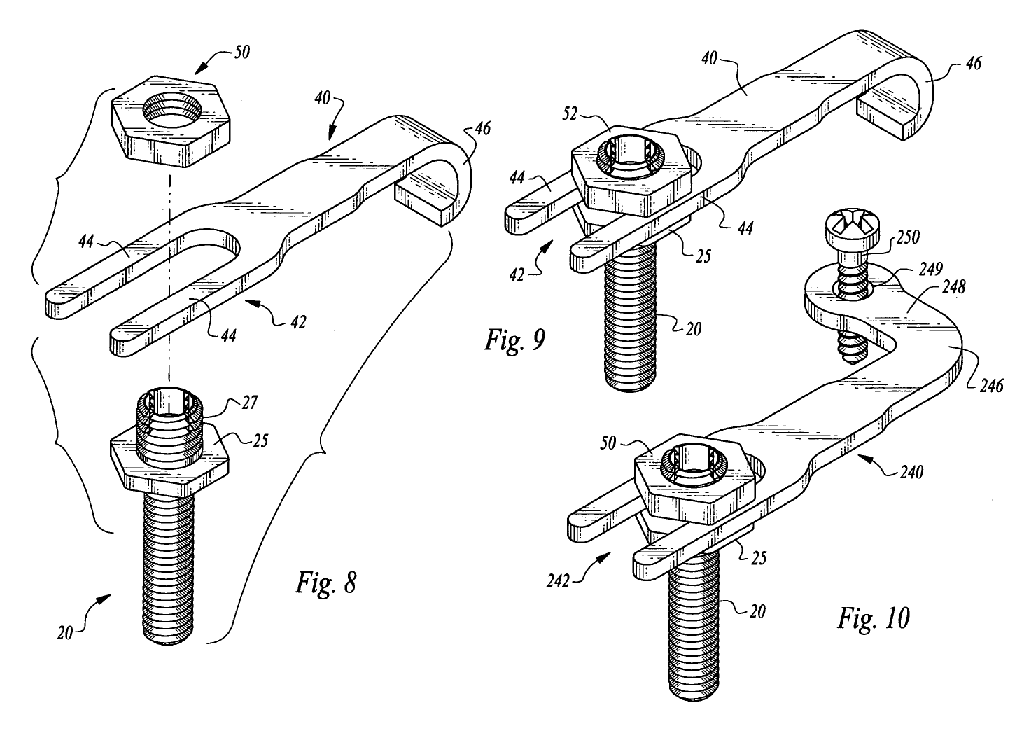

In essence, and with particular reference to FIGS. 8, 9, 12, 17 and 20, basic details of the fixation system 10 of this invention are described, according to a preferred embodiment. The fixation system 10 is generally made up of three separate elements including a first element preferably in the form of a translaminar cannula 20, a second element preferably in the form of a bracket 40 and a third element preferably in the form of a translaminar facet screw 60. The translaminar cannula 20 is an elongate rigid structure which is preferably threaded on an outer surface thereof and includes a central bore 30 extending axially therethrough. A shoulder 25 is also provided at an intermediate exterior location on the translaminar cannula 20 in the preferred embodiment.

The bracket 40 includes a first end with a yoke 42 or similar structure which can straddle the translaminar cannula 20 adjacent the shoulder 25. A nut 50 can be secured to the translaminar cannula 20 to capture the first end of the bracket 40 between the nut 50 and the shoulder 25 to secure the first end of the bracket 40 to the translaminar cannula 20. The second end of the bracket 40 preferably includes a hook 46 thereon. This hook 46 can be secured to the inferior vertebra IV, such as by wrapping around the ipsilateral lamina IL of the inferior vertebra IV. As an alternative (FIGS. 10 and 18) the second end of the bracket 240 can be formed with a hole 249 to receive a pedicle screw 250 or other fastener therein for securing the second end of the bracket 240 to the inferior vertebra IV.

The translaminar facet screw 60 is an elongate rigid structure sized to fit within the central bore 30 of the translaminar cannula 20 and long enough to pass entirely through the translaminar cannula 20 and out of the translaminar cannula 20 to engage portions of the vertebrae beyond the translaminar cannula 20. Most preferably, the translaminar cannula 20 is aligned passing through the contralateral lamina CL of the superior vertebra SV so that the translaminar facet screw 60 passes through the contralateral facet joint including the superior facet SF and the inferior facet IF to fix the contralateral facet joint and fix the superior vertebra SV to the inferior vertebra IV at the contralateral facet joint. The head 62 of the translaminar facet screw 60 engages the translaminar cannula 20, preferably within portions of the central bore 30, so that the translaminar facet screw 60 draws the inferior vertebra IV up against the superior vertebra SV as the translaminar facet screw 60 is tightened.

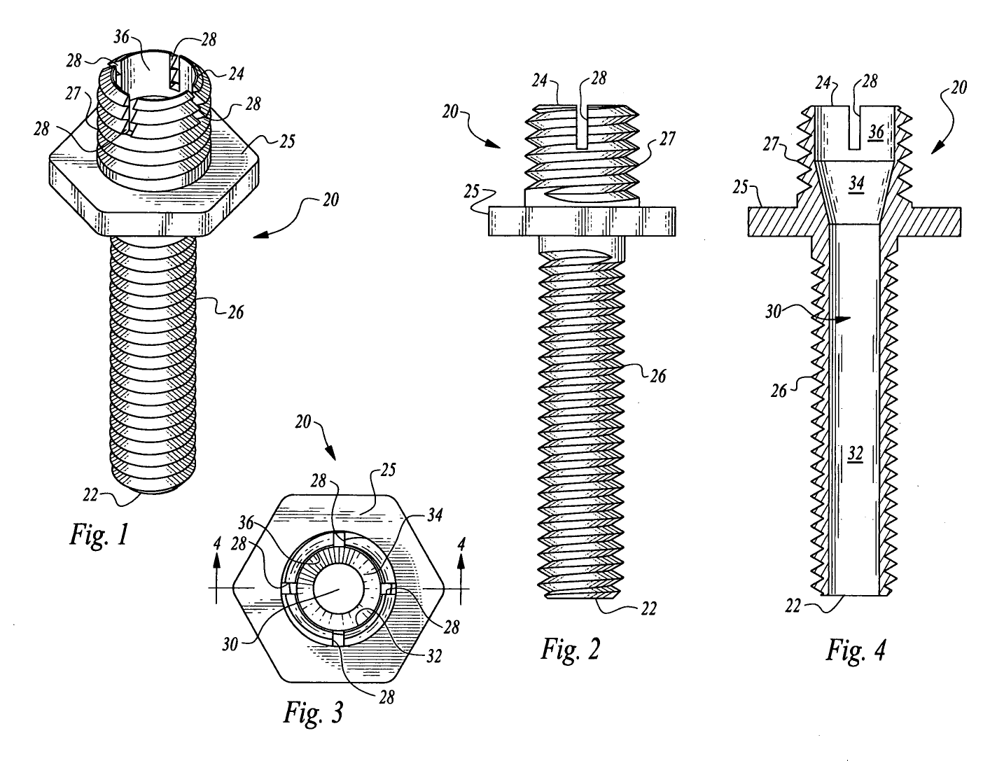

More specifically, and with particular reference to FIGS. 1-4, basic details of the translaminar cannula 20, providing a preferred form of the first element of the fixation system 10 of this invention, are described. The translaminar cannula 20 is a generally cylindrical elongate rigid construct formed of a biocompatible material, such as stainless steel or titanium (or alloys thereof). The translaminar cannula 20 is preferably threaded on an outer surface with shaft threads 26 to allow the translaminar cannula 20 to be readily fixed within a bore formed in the superior vertebra SV.

The translaminar cannula 20 extends from a distal end 22 to a proximal end 24. The shoulder 25 is located between the distal end 22 and proximal end 24, and preferably closer to the proximal end 24. The shoulder 25 preferably abuts a surface of the superior vertebra SV when the translaminar cannula 20 has been implanted into a bore formed in the superior vertebra SV. The shoulder 25 preferably includes facets thereon so that the shoulder 25 not only acts as a stop to prevent the translaminar cannula 20 from being implanted too deeply into the superior vertebra SV, but also acts as a structure through which torque can be applied to the translaminar cannula 20 to cause rotation and insertion into the bore in the superior vertebra SV (along arrow B of FIG. 16).

The translaminar cannula 20 preferably has a different diameter on a proximal side of the shoulder 25 than on a distal side of the shoulder 25. The proximal side of the shoulder 25 is preferably characterized by having a greater diameter than the distal side. Head threads 27 are preferably provided on the proximal side of the shoulder 25. These threads 27 are not required to engage bone and so might have a shallower pitch and otherwise be constructed for receipt of the nut 50 (FIG. 8), rather than for engagement with bone.

Slots 28 are preferably formed in the portions of the translaminar cannula 20 on the proximal side of the shoulder 25. These slots 28 extend axially and provide a second means by which torque can be applied to the translaminar cannula 20, such as through a plain screwdriver or other torque applying tool to rotate the translaminar cannula 20 and cause it to be implanted into a bore in the superior vertebra SV. These slots 28 preferably divide the proximal end 24 of the translaminar cannula 20 into at least four separate quadrants. As described in detail below, this facilitates the flaring of the proximal end 24 of the translaminar cannula 20 radially outwardly (along arrow F of FIG. 13) such as when abutted by the translaminar facet screw 60.

The translaminar cannula 20 has a central bore 30 passing entirely axially therethrough from the distal end 22 to the proximal end 24 (FIG. 4). This central bore 30 is sized to allow the translaminar facet screw 60 to pass entirely therethrough (FIGS. 12 and 19, along arrows D and E). The central bore 30 is preferably smooth and includes a cylindrical portion 32 defining a majority of the central bore 30 and a portion of the central bore 30 closest to the distal end 22. The central bore 30 also includes a frustoconical portion 34 which tapers to a larger diameter in a proximal direction away from the cylindrical portion 32. A head portion 36 is provided on a proximal side of the frustoconical portion 34. This head portion 36 is also preferably cylindrical in form. The head portion 36 has a greater diameter than the cylindrical portion 32.

This contour for the central bore 30 allows portions of the translaminar facet screw 60 to abut the translaminar cannula 20 while recessed entirely within the central bore 30. Such abutment allows the translaminar cannula 20 and associated superior vertebra SV to be drawn tightly toward the inferior vertebra IV when the translaminar facet screw 60 is engaging the inferior vertebra IV at the contralateral facet joint and is further rotated while in abutment within the central bore 30 of the translaminar cannula 20.

While the translaminar cannula 20 provides a preferred form for the first element, the first element could have a variety of different alternative configurations and still function as a basic form of first element for the fixation system 10 of this invention. For instance, the shaft threads 26 are not strictly required, but some other method could be utilized for fixing the translaminar cannula 20 within a bore in the superior vertebra SV. For instance, other fasteners could be utilized to fasten the translaminar cannula 20 in position within the superior vertebra SV or biocompatible adhesives could be utilized or the translaminar cannula could be fitted with other fasteners for securing the translaminar cannula to the superior vertebra SV.

While the first element preferably includes a central bore such that it acts as a form of cannula, the first element could merely act as a guide for the translaminar facet screw 60 or merely be a structure attachable to the translaminar facet screw and to which the bracket 40 or other compression member attaches. In a simple form the first element could merely be an anchor fastened to the superior vertebra to which the second element such as the bracket 40 could attach and to which the third element such as the translaminar facet screw 60 could attach.

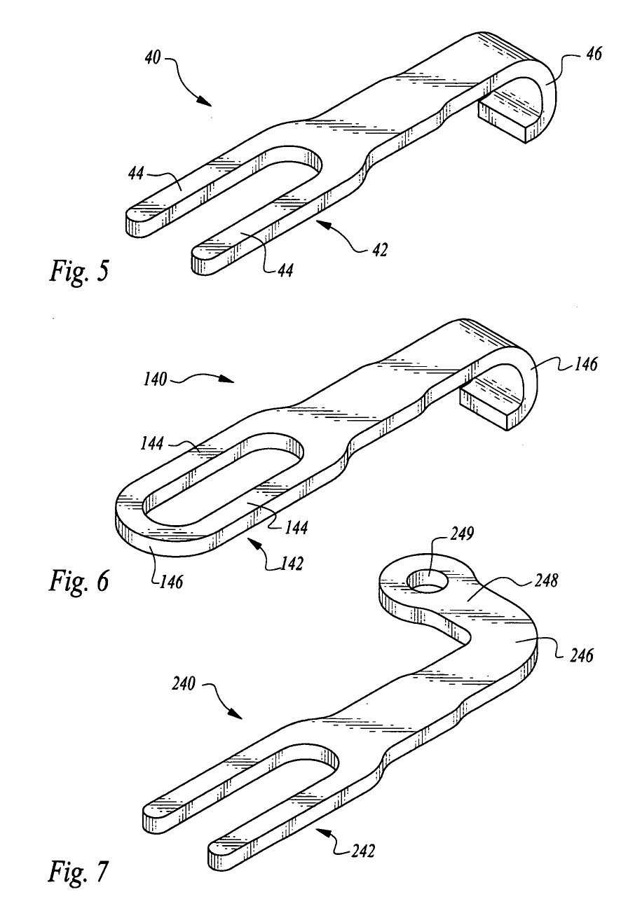

With particular reference to FIGS. 5-10, basic details of the bracket 40, providing a preferred form of second element for the fixation system 10, are described. The bracket 40 provides a preferred form of compression member which acts to join the superior vertebra SV to the inferior vertebra IV on an ipsilateral side of the spine. This bracket 40 includes a first end opposite a second end. The first end is configured to be attached to the translaminar cannula 20. This first end preferably is in the form of a yoke 42 formed of a pair of parallel arms 44 extending away from other portions of the bracket 40. A hook 46 is provided at a second end of the bracket 40 opposite the yoke 42. The yoke 42 is sized to straddle the translaminar cannula 20 on the proximal side of the shoulder 25 and abutting the shoulder 25. A nut 50 can be provided which threads onto the head threads 27 on the proximal side of the shoulder 25 on the translaminar cannula 20. This nut 50 can capture the arms 44 of the yoke 42 adjacent the shoulder 25 to secure the bracket 40 to the translaminar cannula 20.

An alternative bracket 140 is shown in FIG. 6 including a collar 142 in place of the yoke 42. Arms 144 similar to the arms 44 are joined together in an arch 146 so that a complete collar 142 is provided. Such a collar 142 can surround the translaminar cannula 20 and be loosely captured by the nut 50 when the nut 50 is on the head threads 27 of the translaminar cannula 20, but not yet tight. Such a configuration can aid in avoiding undesirable displacement of the alternative bracket 140 away from the translaminar cannula 20, such as during the implantation procedure. A hook 148 is provided on the alternative bracket 140 similar to the hook 46 of the bracket 40.

A second alternative bracket 240 is also provided (FIGS. 7, 10 and 18). In this second alternative bracket 240, a yoke 242 is provided similar to the yoke 42 of the bracket 40. The second end of the second alternative bracket 240 features a bend 246 followed by a tab 248 ending at a hole 249. This hole 249 can receive a pedicle screw 250 therein to secure the second end of the second alternative bracket 240 to the pedicle of the inferior vertebra IV. A pedicle nut 255 (FIG. 18) can be used if the pedicle screw is provided as a threaded shaft without a head.

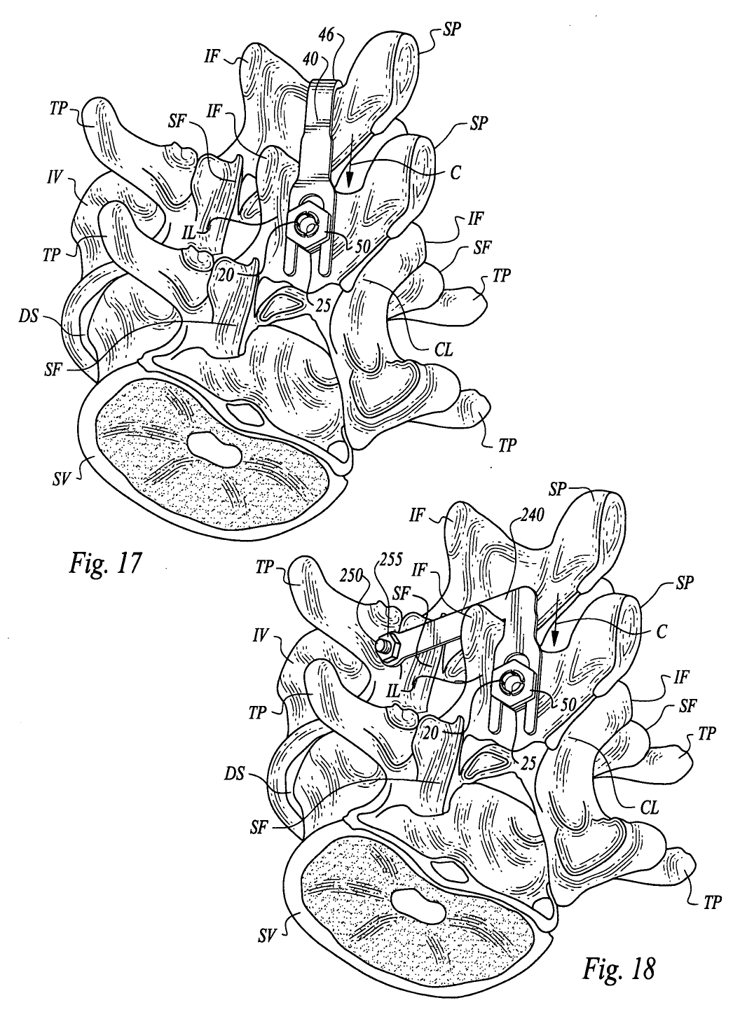

By strategically configuring the angle of the bend 246 and the length of the tab 248, this hole 249 can either be placed over a pedicle of the inferior vertebra IV or over the ipsilateral lamina IL of the inferior vertebra IV, or some other portion of the inferior vertebra IV, where fixation with a screw or other fastener passing through the hole 249 can be accomplished. The bracket 40 remains on an ipsilateral side of the superior vertebra SV and the inferior vertebra IV (FIGS. 17 and 18). Thus, the bracket 40 provides for fixation between the superior vertebra SV and the inferior vertebra IV on the ipsilateral side of the spine (by compression along arrow C of FIGS. 17 and 18).

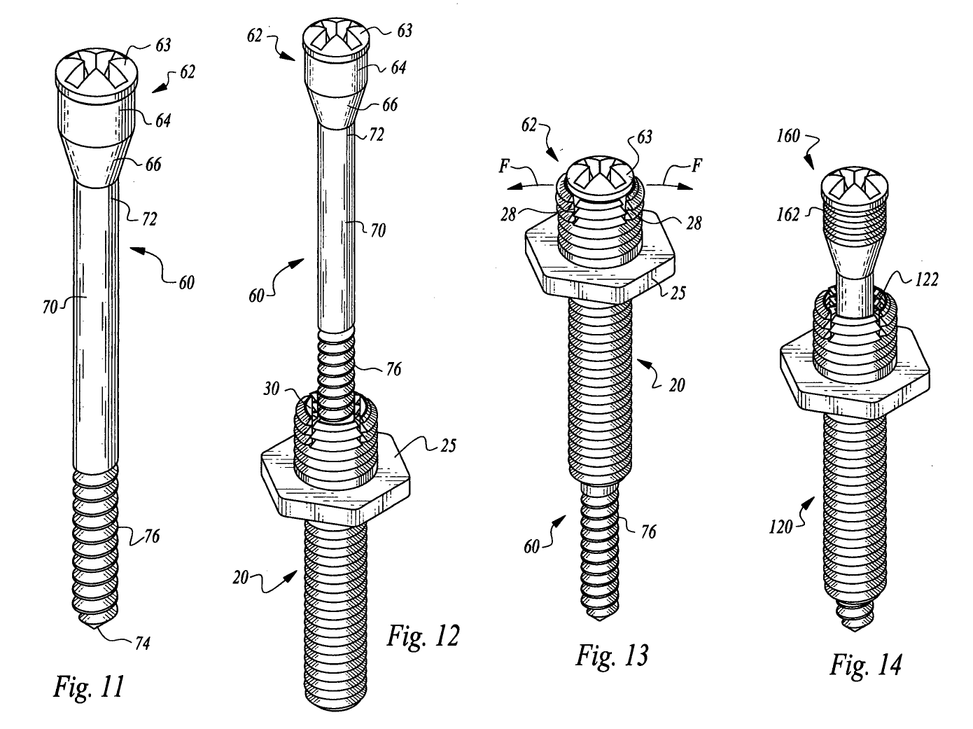

With particular reference to FIGS. 11-14, details of the translaminar facet screw 60, providing a preferred form of third element of the fixation system 10, are described. The translaminar facet screw 60 is a rigid elongate structure formed of biocompatible material such as stainless steel or titanium, or various alloys thereof. The translaminar facet screw 60 is elongate and linear in form extending from a head 62 to a tip 74.

The head 62 preferably includes a slotted face 63 which can receive a torque applying tool, such as a screwdriver, within slots in the slotted face 63. The head 62 includes a cylindrical side wall 64 having a diameter similar to but preferably slightly less than a diameter of the head portion 36 of the central bore 30 of the translaminar cannula 20. The head 62 can thus reside within the head portion 36 of the central bore 30.

A frustoconical side wall 66 extends from the cylindrical side wall 64 toward the tip 74. This frustoconical side wall 66 acts as a tapered portion of the translaminar facet screw 60 which can engage the frustoconical portion 34 of the central bore 30 (FIG. 4). At a minimum, greatest diameter portions of the frustoconical side wall 66 of the translaminar facet screw 60 are greater in diameter than least diameter portions of the frustoconical portion 34 of the central bore 30 on the translaminar cannula 20. In this way, as the translaminar facet screw 60 passes through the central bore 30 (FIGS. 12 and 19, along arrow D), the frustoconical side wall 66 of the translaminar facet screw 60 abuts the frustoconical portion 34 of the central bore 30 within the translaminar cannula 20.

A shank 70 extends linearly from the frustoconical side wall 66 to the tip 74. The shank 70 has a head end 72 adjacent the frustoconical side wall 66 and opposite the tip 74. The shank 70 preferably is characterized by having a substantially constant diameter and linear form with a length greater than a distance through the central bore 30 on the translaminar cannula 20, so that the tip 74 extends out of the distal end 22 of the translaminar cannula 20 where it can engage the contralateral facet joint and in particular the superior facet SF of the inferior vertebra IV at the contralateral facet joint.

The tip 74 includes threads 76 thereon as a preferred form of means to engage bone of the inferior vertebra. These threads 76 are preferably either self tapping or include a portion thereof which acts as a tapping bit as the translaminar facet screw 60 is rotated into position. As an alternative, a separate tapping tool could be provided through the translaminar cannula 20 before final implantation of the translaminar facet screw 60.

As the threads 76 engage bone within the contralateral facet joint, the translaminar facet screw 60 is drawn into the central bore 30 until the frustoconical side wall 66 of the translaminar facet screw 60 abuts the frustoconical portion 34 of the central bore 30 of the translaminar cannula 20. At which time, further rotation of the translaminar facet screw 60 causes the superior facet SF of the inferior vertebra IV to be drawn toward the inferior facet IF of the superior vertebra SV at the contralateral facet joint, closing the contralateral facet joint tight and the superior vertebra SV tightly against the inferior vertebra IV for fixation of the two vertebra SV, IV together on a contralateral side of the spine.

Most preferably, such further rotation of the translaminar facet screw 60, after closing the facet joint on the contralateral side of the spine, causes some deformation of the translaminar cannula 20 at the proximal end 24 thereof. Such deformation is in the form of applying a radial outward force (along arrow F of FIG. 13). Such flaring outward of this proximal end 24 of the translaminar cannula 20 acts to retain the nut 50 against the bracket 40 and shoulder 25, such that the translaminar facet screw 60 helps to ensure that the bracket 60 remains tightly in place fixing the ipsilateral side of the superior vertebra SV to the inferior vertebra IV.

With particular reference to FIG. 14, an alternative threaded translaminar cannula 120 is described. In this alternative translaminar cannula 120 inside threads 122 are provided adjacent the proximal end. These inside threads 122 are sized to engage with outside threads 162 on an alternative translaminar facet screw 160. These inside threads 122 and outside threads 162 can further help to secure the alternative translaminar facet screw 160 to the translaminar cannula 120. These inside threads 122 and outside threads 162 can be differential threads which grow increasingly tighter as the translaminar facet screw 160 advances within the translaminar cannula 120, or can merely have a pitch distinct from that of the threads 76 adjacent the tip 74 on the translaminar facet screw 160 so that engagement and tight capture of the translaminar facet screw 60 to the translaminar cannula 20 is accomplished.

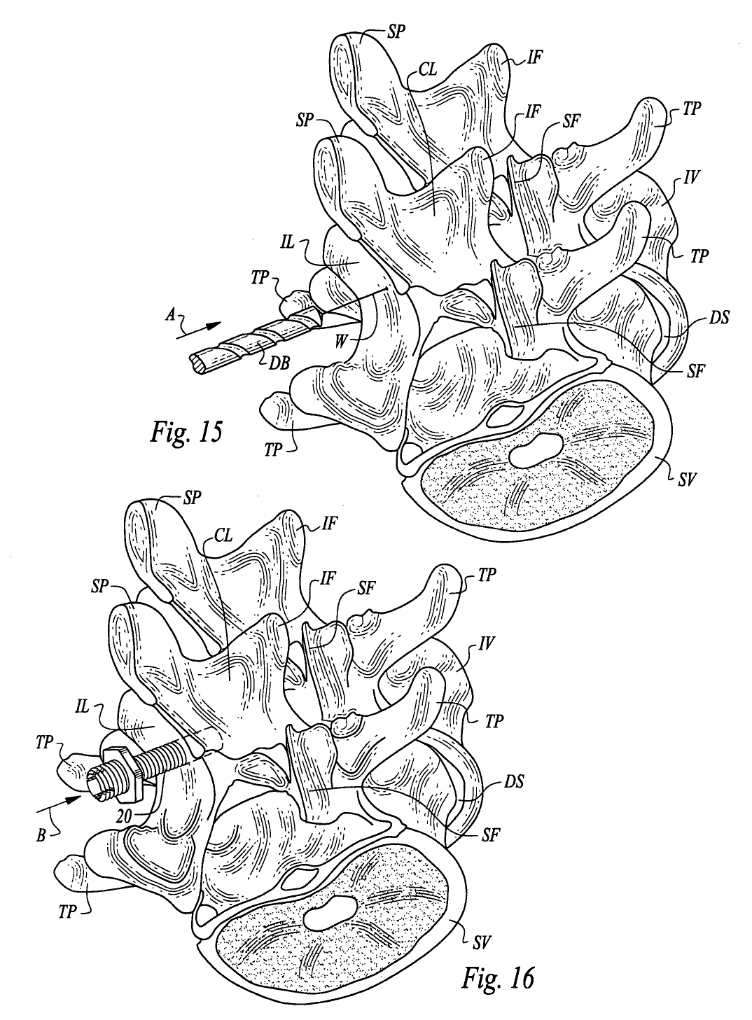

In use and operation, and with particular reference to FIGS. 15-20, details of the method of implantation of the fixation system 10 of this invention are described according to the preferred embodiment. Initially, an incision is made on a posterior lateral position relative to the spine. Such an incision provides access generally along arrow A of FIG. 15 to the superior vertebra SV. A guide wire W can initially be placed along a path desired for a bore into which the translaminar cannula 20 will be implanted. This wire W can be guided through the assistance of a fluoroscope or other imaging device.

Once the guide wire W is in place, a cannulated drill bit DB can be advanced (along arrow A of FIG. 15) to bore a cylindrical hole into the superior vertebra SV. This hole preferably extends from the ipsilateral lamina IL in a direction penetrating the surface of the ipsilateral lamina IL, and then extending within the contralateral lamina CL along a path that remains internal within the contralateral lamina CL and extending toward the contralateral facet joint between the inferior facet IF of the superior vertebra SV and the superior facet SF of the inferior vertebra IV. Preferably, this bore either stops at the contralateral facet joint by only passing through the inferior facet IF of the superior vertebra SV, or stops as a blind bore entirely within the superior vertebra SV.

After this bore has been formed, the threaded translaminar cannula 20 can be passed axially (along arrow B of FIG. 16) into the hole formed in the superior vertebra SV. Such implantation occurs until the shoulder 25 abuts the ipsilateral lamina IL surface on the superior vertebra SV. Note that the placement of the wire W, utilization of the drill bit DB (FIG. 15) and the implantation of the translaminar cannula 20 all occur along a common axis which can occur through a small incision of a posterior lateral nature aligned with a long axis of the translaminar cannula 20.

The bracket 40 or other compression member is then utilized to compress the inferior vertebra IV against the superior vertebra SV, or at least to make preparation for such compression when indicated during the final fixation procedure. Initially, the bracket 40 is placed through the incision and with the hook 46 on the second end of the bracket 40 hooked under the ipsilateral lamina IL of the inferior vertebra IV. The first end of the bracket 40 is extended towards the proximal end 24 of the translaminar cannula 20 with the arms 44 of the yoke 42 straddling the proximal end 24 of the translaminar cannula 20 and against the shoulder 24. The nut 50 is then threaded onto the head threads 27 on the translaminar cannula 20 to secure the first end of the bracket 40 to the translaminar cannula 20 (and compression along arrow C of FIGS. 17 and 18).

Before such fixation of the bracket 40 to the translaminar cannula 20, by tightening of the nut 50, various different surgical procedures can be accomplished, depending on the needs of the surgeon and the particular fixation procedure being performed. For instance, if it is desirable during performance of various activities within the disk space DS to have the inferior vertebra IV drawn tight against the superior vertebra SV, the two vertebra SV, IV can be drawn together with an appropriate reduction clamp, and then the nut 50 tightened so that the bracket 40 keeps the inferior vertebra IV compressed against the superior vertebra SV.

If it is preferable that the vertebra SV, IV be first spaced apart for a surgical procedure to be performed, the nut 50 would not be initially tightened, but rather some form of spacer might be utilized to provide the spacing required between the adjacent vertebra SV, IV. When such spacing requiring procedures are completed, and compression is desired between the adjacent vertebra SV, IV, the vertebra SV, IV would be drawn together and then the nut 50 tightened to secure the bracket 40 to the translaminar cannula 20. Once such tightening of the nut 50 and securing of the bracket 40 occurs, the ipsilateral side of the overall fixation system 10 is complete and the ipsilateral side of the spine has been fixed. The placement of the bracket 40 can occur with an incision on the same posterior lateral side of the fixation site (preferably the same incision is used to implant the translaminar cannula 60).

Finally, it is desirable to add fixation to the contralateral side of the spine. To do so, the translaminar facet screw 60 is passed through the translaminar cannula 20 (along arrow D of FIG. 19). The translaminar facet screw 60 is advanced, either by tapping its own hole through the contralateral facet joint or by first pre-drilling and/or tapping a hole passing through the contralateral facet joint. The translaminar facet screw 60 then passes through the contralateral facet joint including the superior facet SF of the inferior vertebra IV in the inferior facet IF of the superior vertebra SV (along arrow E of FIG. 20). Threads 76 of the translaminar facet screw 60 engage the superior facet SF of the inferior vertebra IV, drawing the superior facet SF against the inferior facet IF and fixing the inferior vertebra IV to the superior vertebra SV on the contralateral side of the spine. Thus, the translaminar facet screw 60, by passage along arrow E of FIG. 20, provides fixation on the contralateral side of the spine. Note that the translaminar facet screw 60 is advanced and manipulated entirely from the ipsilateral side of the spine, and so can be accessed through the common incision through which the translaminar cannula 20 was implanted.

As the translaminar facet screw 60 is tightened, eventually the frustoconical side wall 66 of the translaminar facet screw 60 abuts the frustoconical portion 34 of the central bore 30 within the translaminar cannula 20, so that the superior vertebra SV and inferior vertebra IV are drawn together. Furthermore, the dimensions of the head 62 on the translaminar facet screw 60 and dimensions of the head portion 36 of the central bore 30 are preferably such that the proximal end 24 of the translaminar cannula 20 can flare outwardly (along arrow F of FIG. 13) in a radial direction through final tightening of the translaminar facet screw 60. Such flaring of the proximal end 24 of the translaminar cannula 20 causes the translaminar cannula 20 to retain the nut 50, tightly securing the bracket 40 to the shoulder 25 of the translaminar cannula 20.

This disclosure is provided to reveal a preferred embodiment of the invention and a best mode for practicing the invention. Having thus described the invention in this way, it should be apparent that various different modifications can be made to the preferred embodiment without departing from the scope and spirit of this invention disclosure. When structures are identified as a means to perform a function, the identification is intended to include all structures which can perform the function specified. When structures of this invention are identified as being coupled together, such language should be interpreted broadly to include the structures being coupled directly together or coupled together through intervening structures. Such coupling could be permanent or temporary and either in a rigid fashion or in a fashion which allows pivoting, sliding or other relative motion while still providing some form of attachment, unless specifically restricted.

What is claimed is:

1. A system for fixation of two adjacent spinal vertebrae, including a superior vertebra and an inferior vertebra through a posterior or posterior-lateral incision, the system comprising in combination:
an elongate translaminar cannula having a hollow central bore passing from a proximal end to a distal end;
said translaminar cannula adapted to be fixed to the superior vertebra with a proximal end extending from the ipsilateral lamina of the superior vertebra and with a distal end extending through the contralateral lamina of the superior vertebra toward the contralateral facet joint between the superior vertebra and the inferior vertebra;
a compression member having a first end adapted to be secured to said translaminar cannula adjacent where said translaminar cannula is adapted to extend from the ipsilateral lamina of the superior vertebra and having a second end adapted to be secured to an ipsilateral portion of the inferior vertebra; and
a translaminar facet screw having a head opposite a tip and with threads adjacent said tip, said translaminar facet screw longer than said translaminar cannula and with portions thereof at least as small in diameter as said bore in said translaminar cannula, said translaminar facet screw long enough to pass through said bore of said translaminar cannula when said translaminar cannula is located with said proximal end extending from the ipsilateral lamina of the superior vertebra and with the distal end extending through the contralateral lamina of the superior vertebra toward the contralateral facet joint, with said threads of said translaminar facet screw crossing the contralateral facet joint between the superior vertebra and the inferior vertebra, said threads adapted to engage at least the inferior vertebra to join the superior vertebra and the inferior vertebra together at the contralateral facet joint.

2. The system of claim 1 wherein said translaminar cannula includes threads on an outer surface thereof, said threads adapted to engage the superior vertebra to at least partially fix said translaminar cannula to the superior vertebra.

3. The system of claim 1 wherein said compression member includes a hook at said second end, said hook adapted to wrap around a portion of the inferior vertebra when said first end of said compression member is secured to said translaminar cannula fixed to the superior vertebra.

4. The system of claim 3 wherein said translaminar cannula has a shoulder closer to said proximal end of said translaminar cannula than to said distal end of said translaminar cannula, said translaminar cannula including head threads between said shoulder and said proximal end, said first end of said compression member adapted to abut said shoulder on a side of said shoulder closest to said proximal end of said translaminar cannula, said first end of said compression member secured to said translaminar cannula with a nut threaded on said head threads between said shoulder and said proximal end.

5. The system of claim 4 wherein said compression member includes a yoke at said first end including a pair of arms spaced apart by a width at least as great as a diameter of said translaminar cannula between said shoulder and said proximal end, said yoke adapted to be trapped between said shoulder and said nut to secure said compression member to said translaminar cannula.

6. The system of claim 4 wherein said compression member includes a collar at said first end, said collar including a pair of arms spaced apart by a width at least as great as a diameter of said translaminar cannula between said shoulder and said proximal end, said arms joined together at ends thereof by an arch such that said first end of said compression member forms a collar surrounding said translaminar cannula adjacent said shoulder and between said shoulder and said proximal end, with said collar of said compression member adapted to be secured to said translaminar cannula between said nut and said shoulder.

7. The system of claim 1 wherein said compression member includes a hole passing through said second end, said second end spaced from said first end by a distance similar to a distance from the ipsilateral lamina of the superior vertebra where said first end is adapted to be secured to said translaminar cannula, to the pedicle of the inferior vertebra, such that said hole in said second end of said compression member is aligned with the pedicle of the inferior vertebra when said first end of said compression member is secured to said translaminar cannula with said translaminar cannula fixed to the superior vertebra at the ipsilateral lamina of the superior vertebra; and
said compression member including a pedicle screw passing through said hole, said pedicle screw adapted to thread into the pedicle of the inferior vertebra to secure said second end of said compression member to the inferior vertebra.

8. The system of claim 1 wherein said bore in said translaminar cannula includes at least a portion thereof which tapers from a greater diameter adjacent said proximal end to a lesser diameter as said bore extends towards said distal end, said translaminar facet screw having at least a portion thereof between said head and said tip which tapers from a greater diameter closer to said head to a lesser diameter closer to said tip, said tapering portion of said translaminar facet screw sized to abut said tapering portion of said translaminar cannula when said translaminar facet screw is passed through said bore from said proximal end of said translaminar cannula towards said distal end.

9. The system of claim 8 wherein said translaminar cannula includes slots extending longitudinally into said proximal end of said translaminar cannula, said slots adapted to allow portions of said translaminar cannula adjacent said proximal end to flare radially outwardly when radial outward forces are applied to said proximal end of said translaminar cannula, such as by abutment of said tapering surface of said translaminar facet screw with said tapering surface of said bore of said translaminar cannula.

10. The system of claim 1 wherein said bore of said translaminar cannula includes threads on a portion thereof adjacent said proximal end of said translaminar cannula, said translaminar facet screw including threads on an outer surface of said head, said threads on said translaminar facet screw head adapted to engage threads within said bore of said translaminar cannula to join said translaminar facet screw securely to said translaminar cannula.

11. A system for fixation of two adjacent spinal vertebra, including a superior vertebra and an inferior vertebra, the system comprising in combination:
a first element adapted to be fixed to the superior vertebra on the ipsilateral side of the spinous process;
a second element having a first end adapted to be affixed to said first element;
said second element having a second end spaced from said first end, said second end adapted to be secured to an ipsilateral portion of the inferior vertebra;
a third element having an elongate form between a head and a tip, with a means to engage bone adjacent said tip, said third element sufficiently long to extend from the ipsilateral side of the spinous process, through the contralateral lamina of the superior vertebra and through the contralateral facet joint between the superior vertebra and the inferior vertebra; and
said third element adapted to be secured to said first element while said third element is extending from the ipsilateral side of the spinous process of the superior vertebra through the contralateral lamina of the superior vertebra and through the contralateral facet joint between the superior vertebra and the inferior vertebra.

12. The system of claim 11 wherein said first element includes a bore adapted to be aligned with the contralateral lamina and the contralateral facet joint with a proximal end of the bore open to an ipsilateral side of the spinous process, said bore adapted to receive said third element passing therethrough with portions of said third element adjacent said tip passing beyond said bore and adapted to engage portions of the inferior vertebra adjacent the contralateral facet joint between the superior vertebra and the inferior vertebra.

13. The system of claim 12 wherein said first element includes a shoulder, said first element having an elongate form between a proximal end and a distal end, said shoulder located closer to said proximal end than to said distal end, said shoulder adapted to be coupled to said first end of said second element.

14. The system of claim 13 wherein said second element includes arms on said first end, said arms spaced apart by a width at least as great as a width of the first element between said shoulder and said proximal end of said first element, and a nut removably attachable to said first element between said proximal end and said shoulder, said nut adapted to capture said arms of said first end of said second element between said nut and said shoulder to hold said second element to said first element.

15. The system of claim 14 wherein said arms are opened at ends thereof most distant from said second end, such that said first end of said second element is in the form of a yoke.

16. The system of claim 14 wherein said arms of said second element are joined together at said first end such that said arms form a portion of a collar at said first end of said second element.

17. The system of claim 12 wherein said first element includes threads on an outside surface of said first element, said first element having an elongate form adapted to pass through said contralateral lamina in an elongate direction, and remaining substantially within an interior of said contralateral lamina with said bone extending coaxially therein, said threads on said outside of said first element adapted to engage the superior vertebra to secure said first element as a threaded translaminar cannula.

18. The system of claim 1I wherein said bone engagement means of said third element includes threads adjacent said tip adapted to engage bone at the contralateral facet joint to secure the superior vertebra and the inferior vertebra at the contralateral facet joint.

19. The system of claim 12 wherein said bore of said first element includes at least a portion thereof which tapers from a greater diameter to a lesser diameter as said first element extends from a proximal end to a distal end, said third element having at least a portion thereof which tapers between said head and said tip, said third element having a diameter which causes said tapering portion of said third element to abut said tapering portion within said bore of said first element, said third element adapted to engage said first element at said tapering portion while also engaging bone of said inferior vertebra at said tip, such that said inferior vertebra and said superior vertebra are fixed securely together.

20. The system of claim 19 wherein said first element includes portions adjacent said proximal end thereof which are adapted to flare radially outwardly.

21. The system of claim 20 wherein said first element includes slots adjacent said proximal end splitting said first element into separate segments which can flex radially outwardly away from each other when radial outward forces are applied thereto.

22. A method for fixation of two adjacent spinal vertebrae, including a superior vertebra and an inferior vertebra, through a posterior-lateral incision, the method including the steps of:
providing a kit for fixation of the two adjacent spinal vertebrae including a first element adapted to be fixed to the superior vertebra on the ipsilateral side of the spinous process; a second element having a first end adapted to be affixed to the first element; the second element having a second end spaced from the first end, the second end adapted to be secured to an ipsilateral portion of the inferior vertebra; a third element having an elongate form between a head and a tip, with a means to engage bone adjacent the tip, the third element sufficiently long to extend from the ipsilateral side of the spinous process, through the contralateral lamina of the superior vertebra and through the contralateral facet joint between the superior vertebra and the inferior vertebra; and the third element adapted to be secured to the first element while the third element is extending from the ipsilateral side of the spinous process of the superior vertebra through the contralateral lamina of the superior vertebra and through the contralateral facet joint between the superior vertebra and the inferior vertebra;
coupling the first element to the superior vertebra on the ipsilateral side of the spinous process on the superior vertebra;
securing the second end of the second element to an ipsilateral portion of the inferior vertebra;
affixing the first end of the second element to the first element; and
placing the third element through the contralateral lamina and through the contralateral facet joint with the third element secured to the first element and with the superior vertebra fixed to the inferior vertebra at the contralateral facet joint by the third element.

23. The method of claim 22 including the further steps of:
forming the first element to be elongate in form with a hollow bore passing from a proximal end to a distal end, said first element including threads on an outside surface thereof adapted to engage bone within the contralateral lamina to secure the first element to the superior vertebra within the contralateral lamina of the superior vertebra;
cutting a hole in the superior vertebra having a size similar to that of the first element and extending from the ipsilateral side of the spinous process into the contralateral lamina; and
threading the first element into the hole to secure the first element to the superior vertebra within the hole in the superior vertebra.

24. The method of claim 23 including the further steps of forming the bore within the first element to have at least a portion thereof which tapers from a greater diameter closer to said proximal end to a lesser diameter closer to said distal end;
forming said third element to include at least a portion thereof which tapers in diameter from a diameter greater than a least diameter portion of the bore to a diameter less than a least diameter portion of the bore; and
placing the third element coaxially through the bore in the first element until the third element tapering portion abuts the bore tapering portion within the first element, and with the tip of the third element passing out of the distal end of the first element and engaging the inferior vertebra at the contralateral facet joint when the third element tapered portion abuts the bore tapered portion.

25. The method of claim 22 wherein said coupling step, said securing step, said affixing step and said placing step each occur through a single posterior lateral incision.
